(19) United States
(12) Patent Application Publication
(10) Pub. No.: US 2019/0175808 A1
(43) Pub. Date: Jun. 13, 2019

(54) HYBRID POWERING SYSTEM FOR AN IMPLANTED MEDICAL DEVICE

(71) Applicant: Leviticus Cardio Ltd., Petach Tikva (IL)

(72) Inventors: Michael Zilbershlag, Givat Shmuel (IL); Neri Naveh, Kiryat Ono (IL)

(21) Appl. No.: 16/217,428

(22) Filed: Dec. 12, 2018

Related U.S. Application Data
(60) Provisional application No. 62/635,734, filed on Feb. 27, 2018.
(60) Provisional application No. 62/597,570, filed on Dec. 12, 2017.

(51) Int. Cl. A61M 1/12; A61M 1/10

(52) U.S. Cl. CPC A61M 1/127; A61M 1/1086; A61M 2205/52; A61M 2205/3523; A61M 2205/8206; A61M 1/122

(57) ABSTRACT

A hybrid powering system for an implanted medical device combines wireless power transfer with transcutaneous wired power transfer and/or control. A preferred embodiment for powering a ventricular assist device (VAD) includes an implantable controller with a rechargeable battery, and an implantable power receiver antenna for receiving wireless power from a transmitter located outside of the patient's body. The power receiver charges the battery and allows the controller to drive the VAD. The system also includes the ability to connect a hardwired connection via a connector device configured to be implanted percutaneously. The connector device provides a socket for an external power source or an external controller to plug directly into the system, providing hardwired power and/or control to the implanted VAD. When an external controller is connected it causes the implanted controller to stop driving the VAD, in order to avoid short circuiting the VAD. The percutaneous connector device can be used as a backup power source in case the wireless connection fails, or it can be used discretionally, such as for overnight charging.

### CROSS-REFERENCE TO RELATED APPLICATIONS

This application claims the benefit of and priority to U.S. Provisional Application Ser. No. 62/635,734, filed Feb. 27, 2018, and U.S. Provisional Application Ser. No. 62/597,570, filed Dec. 12, 2017, the contents of each of which are incorporated by reference in their entirety.

### TECHNICAL FIELD

The present disclosure relates to systems for powering implanted medical devices such as ventricular assist devices.

### BACKGROUND

Congestive heart failure is a disease that results from the inability of the heart to pump blood throughout the body at its normal pace, causing blood to flow a slower rate with increased pressure. As a result, the heart is unable to meet the oxygen and nutrient demands of an individual's vital organs. Heart failure may be caused by cardiomyopathy, heart valves damage, coronary heart disease, hypertension, and, in some cases, diabetes. Worldwide, millions of patients currently suffer from congestive heart failure. While many are candidates for heart transplantation, others can be helped with electro-mechanical heart implants, such as a ventricular assist device (VAD), which is an implantable electro-mechanical pump that serves to improve or replace the function of a failing heart.

VADs require a power source to operate the pump. In a traditional arrangement, the VAD is connected to an external power source by a transcutaneous wire. The exit site of the wire is typically in the patient's abdomen. As with any medical device that requires a transcutaneous wire, the exit site is often vulnerable to infection.

Wireless configurations alleviate the infection problem and are less cumbersome than transcutaneous wires, providing the patient with increased mobility and quality of life. One wireless approach is transcutaneous energy transfer (TET), wherein an external energy source is directed toward an implanted energy harvesting device in an arrangement that seeks to minimize radiofrequency (RF) exposure of the patient. Typically the receiver coil is located just under the patient's skin and the transmitter is located just above the skin. Such TET systems are very sensitive to misalignment and movement of the implanted coil. Another drawback of TET systems is that the electromagnetic field density is so high that it can cause heating of the skin and even burns. An alternative to TET is coplanar energy transfer (CET). CET systems include an implanted receiver coil that receives wireless power from transmitter coil external to the body. The transmitter coil completely surrounds the part of the patient's body wherein the receiver coil is implanted. Unlike in TET, the RF energy that is inductively transmitted into the patient's body from the transmitter coil is spread out and not concentrated or focused on a particular area, reducing the risk of heating and burns. And since the transmitter and receiver merely need to be in the same plane, rather than precisely aligned, CET systems do not suffer from the misplacement problems of TET systems.

Although wireless systems reduce the incidence of infection and increase mobility for the patient, they introduce additional safety risks resulting from the lack of a hardwire connection to the power source. If the wireless connection suddenly fails and the implanted device cannot receive power, a patient's life may be at risk if he cannot quickly get help.

### SUMMARY

The present disclosure provides systems for powering and controlling implanted medical devices such as VADs, in a configuration that allows a user to switch between wireless and hardwired connections. These hybrid systems combine the convenience of wireless electromagnetic power transfer with the safety of hardwired power transfer. Embodiments of the disclosed systems include redundancy for the wireless power source, the implanted controller, or both.

The system generally involves a VAD connected to an implantable controller that operates the motor in the VAD, and an implantable power receiver that provides power to the controller and the VAD. The power receiver is configured to wirelessly receive electromagnetic power from a power transmitter that can be located outside the body such that it can deliver power to the power receiver implanted within the body. The external power transmitter is electrically connected to a power source, such as a battery, and may also be connected to an external controller that regulates the power transfer. In addition to the wireless power transfer components, the system also includes a connector device, which is electrically connected to the other implanted components and is configured to be percutaneously implanted in the patient. The connector device may take the form of a socket or an outlet configured to accept a plug that is hardwired to an external power source, which may be the same power source as the wireless transmitter or may be a different power source altogether. When the external power source is plugged into the connector, it completes a circuit from the external power to the VAD, thereby allowing the VAD to be powered by the external power source. There may also be an external controller associated with the plug, which may be the same as the external controller associated with the wireless power transfer or may be a different controller. When connected, the external controller can send a signal to the implanted controller to cause the implanted controller to stop operating the VAD, and the external controller can then take over control of the VAD.

The system therefore includes the components for wireless power transfer (either TET or CET), as well as a backup hardwired power source and/or controller that can transmit power through a wire to the VAD via the percutaneously implanted connector. The percutaneously implanted connector can be used as a backup power source when the wireless power transfer fails, or it can simply be an alternative power source that the patient can selectively use, such as for overnight charging. The hybrid system provides the user with the freedom to decide if and when an implanted medical device receives power wirelessly or through a hardwired connection.

Aspects of the disclosure relate to a system for treating a heart condition in a patient, the system including a VAD, a controller, and a power receiver each configured to be implanted within the body and electrically associated with one another. The implanted power receiver is further configured to wirelessly receive electromagnetic power from a power transmitter disposed external to the patient's body. The system also includes a connector device configured to be implanted percutaneously in the body and hardwired to the controller. The connector device can be hardwired to a power source external to the patient to provide redundancy for the implanted power receiver.

In embodiments, the implanted controller includes a rechargeable battery configured to be charged by one or both of the implanted power receiver and the external power source.

In some embodiments, the system also includes an external monitoring device configured to communicate with the implanted controller wirelessly, such as via a radio frequency in the communication spectrum for medical implants (MICS). The external monitoring device may receive data from the implanted controller indicating the operational status of the VAD. The data may include an alert that the VAD is not receiving power from the implanted power receiver. The data may be shown on a display associated with the external monitoring device.

The connection device may be configured to be implanted percutaneously in any part of the patient's body. In some embodiments, it is configured to be disposed behind the patient's ear. The connection device may include a socket configured to accept a plug that is hardwired to the external power source.

In a related aspect of the invention, a system for treating a heart condition in a patient includes a connector device configured to be implanted percutaneously and hardwired to the VAD. The connector device is configured to be hardwired to a controller external to the patient. The external controller is configured to determine whether the implanted controller is driving the VAD and to provide redundancy for the implanted controller only when the implanted controller is not driving the VAD.

The external controller can be associated with an external power source. The external controller can be configured to communicate a signal to the implanted controller to cause the implanted controller to stop driving the VAD. The signal can be transmitted wirelessly or via a communication line that runs through the power lines that connect the controllers to the VAD. The signal can be sent automatically in response to the external controller being plugged in to the connector device.

### BRIEF DESCRIPTION OF THE DRAWINGS

FIG. 1 shows a system for driving a VAD with a transcutaneous wired power configuration.

FIG. 2 shows a wireless powering system.

FIGS. 3A and 3B show a coplanar energy transfer system.

FIGS. 4A and 4B show a hybrid powering system.

FIG. 5 shows a powering system with a behind-ear connector device.

FIG. 6 shows a hybrid powering system with a behind-ear connection and an external communication device.

FIGS. 7A and 7B show connectivity arrangements between an internal controller and external communication devices.

### DETAILED DESCRIPTION

The disclosed powering system for implanted medical devices such as a ventricular assist device (VAD) includes the components for wireless power transfer (either TET or CET), as well as a hardwired power source and/or controller that can transmit power through a wire to the VAD via the percutaneously implanted connector. The disclosed system is built from components of traditional VAD systems and combines them in new ways to produce unexpected advantages. In particular, the hardwired power source can be used as an emergency backup power source in case the wireless power transfer fails, or it can be used for charging at any other convenient time for the patient, such as overnight charging. The disclosed systems also include wireless monitoring devices that can allow the user to remove any external charging apparatus and be free and unencumbered except for the monitoring device, which may be a wristwatch. The versatility of the disclosed systems gives the patient several options for how best to charge an implanted medical device, based on the particular needs and lifestyle of the patient, and also provides redundancy for implanted wireless power receivers and controllers.

Although the invention is useful for any implanted medical device, it will be described with reference to a VAD in particular. A VAD is an electro-mechanical device that partially or completely replaces the function of a failing heart. Some VADs are intended for short term use, typically for patients recovering from heart attacks or heart surgery, while others are intended for long term use (months to years and in some cases for life), typically for patients suffering from CHF. Unlike artificial hearts, VADs do not require the removal of the patient's heart. VADs are designed to assist either the right (RVAD) or left (LVAD) ventricle. The choice of device depends on the underlying heart disease and the pulmonary arterial resistance which determines the load on the right ventricle. LVADs are most commonly used but when pulmonary arterial resistance is high, right ventricular assist becomes necessary. Long term VADs are normally used to keep patients alive with a good quality of life while they wait for a heart transplant. Many types of VADs are compatible with the presently disclosed powering system. First generation VADs, like the one described in U.S. Pat. No. 4,906,229, emulate the heart by using a pulsatile action where blood is alternately sucked into the pump from the left ventricle then forced out into the aorta. These devices are usually cumbersome and necessitate major surgery for their implantation into the vascular system and for introducing the cannula into the heart ventricle. More recent devices are based on intravascular continuous flow pumps, which can be roughly categorized as either centrifugal pumps, as described in US 2004/0143151, or axial flow impeller driven pumps, as described in U.S. Pat. No. 4,957,504. These second generation VADs have impellers with high flow rate capability and are much smaller than the first generation VADs, but have contacting bearings that suspend the rigid motor. The bearing contacts generally cause undesirable clot formation either inside or around the periphery of the bearings, making these devices unsuitable for long-term use. In these pumps, blood experiences traumatization and damage due to shearing and vortexing into the small gaps between the outer edge of the stator blades and the inner side of the pipe carrying blood. Newer VADs overcome these issues by suspending the impeller in the pump using either hydrodynamic or electromagnetic suspension, thereby decreasing risks of thrombosis or hemolysis. Such pumps are described for example in U.S. Pat. Nos. 6,527,699 and 7,467,929.

The invention provides hardwired redundancy to a wireless VAD system. Generally a VAD system has a controller that includes or is connected to a rechargeable battery. The battery can be charged by a power source, such as a wireless receiver or a hardwired power connection. A completely wireless system (i.e., where the internal controller is wirelessly powered and has no backup wired connection) would be problematic because if any of the wireless powering components were to fail, there is no easy way to connect a hardwired connection. It is therefore preferable for wireless systems to have redundancy for the implanted components. For example, if the connection between the wireless receiver and the controller fails, a redundant power source could be provided for charging the battery. However, if the controller itself fails, then merely having a backup input power source to the controller does not solve the problem. Likewise, if the connection between the controller and the VAD fails, it is important to have a redundancy for the control of the VAD. For traditional VAD systems with external controllers, the simple solution is to just replace the controller by plugging in a new one. But for implanted controllers, redundancy is more complicated because the implanted controller cannot be readily unplugged from the VAD when a backup controller is activated, and connecting two controllers to the VAD simultaneously would cause a short or an explosion. The invention involves systems for providing redundancy to the wireless power source and/or to the controller, which will be described in greater detail below.

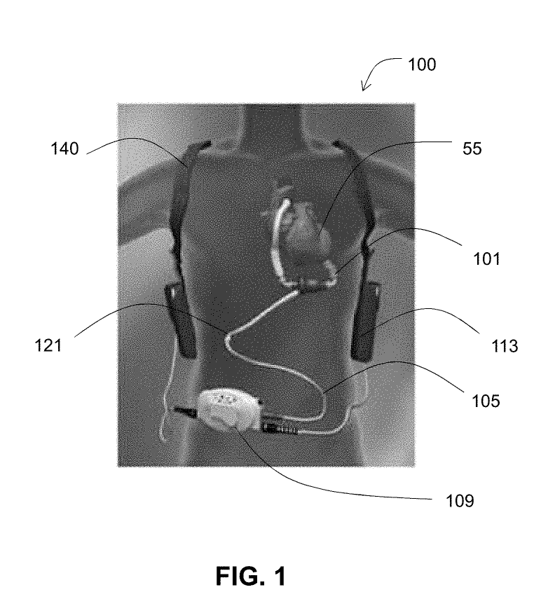

FIG. 1 shows a VAD powering system 100 for driving a VAD 101 with a transcutaneous wired power configuration. The system 100 shows generally how the hardwired powering components of the present invention would work. As with most VADs, a cannula is inserted into the apex of the appropriate ventricle in the heart 55. Blood passes through to a pump and then through a tube to the aorta (in an LVAD) or to the pulmonary artery (in an RVAD). The pump is powered through a wire 105 that extends out of the body through an exit site 121 and is connected to a controller 109 and in turn to a power supply 113. The power supply 113 as shown is a set of rechargeable batteries that can be worn by the patient in a holster 140. As discussed above, traditional VAD systems that rely on transcutaneous wires are prone to infection and can be bulky and cumbersome to the user. The present invention includes a percutaneous connector device (not shown), which will be described in greater detail below.

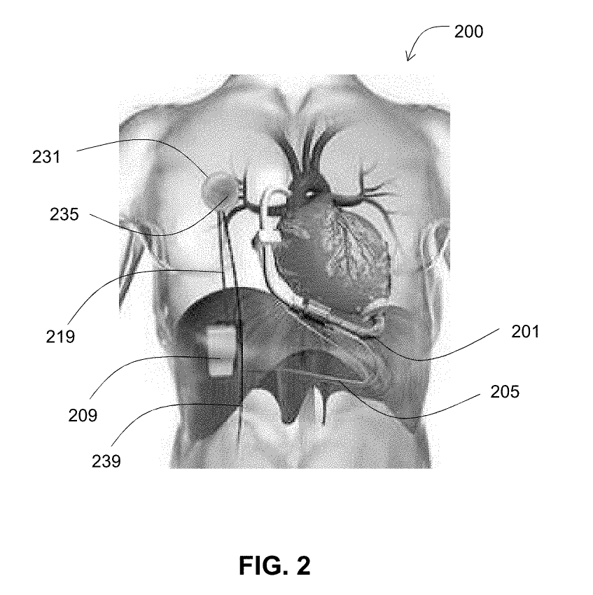

Another benefit of the present disclosure is the inclusion of wireless power, an example of which is shown in FIG. 2. Systems powered solely by wireless power solve some of the aforementioned problems by eliminating the drive line that exits the body. Depending on the capacity of the implanted battery, the patient can typically remove all of the external apparatuses for at least a period of time while the implanted battery is sufficiently charged, providing an additional degree of freedom to the patient. An example of a wireless configuration compatible with the present invention is the TET system shown in FIG. 2. The implanted receiver 231 gets power from an external transmitter 235 via electromagnetic power transfer. In this arrangement, the VAD system 200 can operate without a line going through the patient's skin to connect the implanted device to external power. A drive line 205 is implanted within the patient's body that connects that VAD 201 to an implanted controller 209. The controller 209 will typically also include a battery (not shown) as a temporary power source for the VAD 201. Another drive line 219 implanted within the patient's body connects the implanted controller 209 to an implanted power receiver 231. The implanted power receiver 231 is preferentially located near the surface of the skin, so that it can be aligned with an external power transmitter 235. The external power transmitter 235 is connected by a wire 239 to an external controller (not shown) and external power source such as a battery (not shown). Further examples of TET systems and their operation are described in US 2015/0018600, which is incorporated by reference in its entirety. One disadvantage of a TET system is that the implanted power receiver tends to get heated and can cause discomfort or injury to the patient.

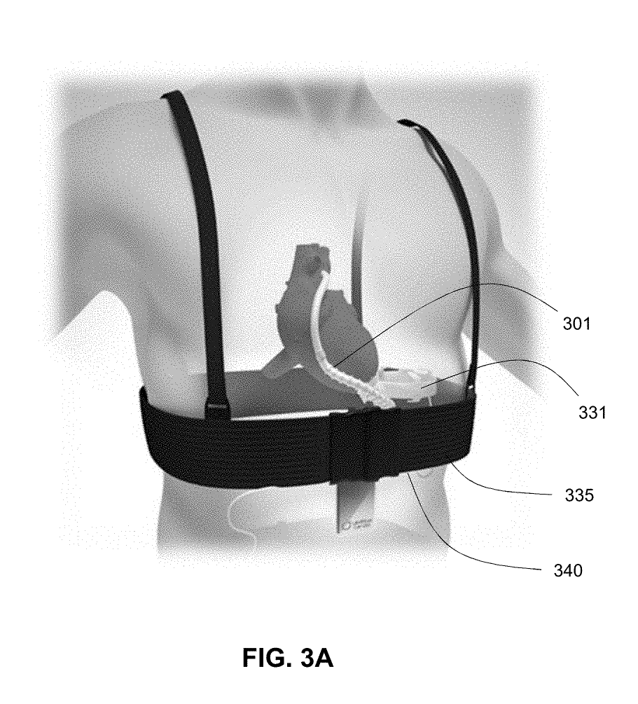

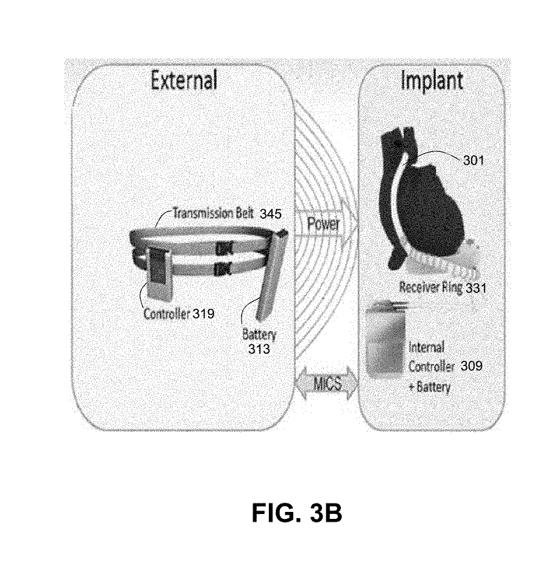

Coplanar energy transfer (CET) systems solve this problem. The CET system shown in FIG. 3A is compatible with the present invention for supplying wireless power. The CET system includes an external transmitter inductive coil 335 that can be provided as a belt 344 designed to be placed externally around a part of a body of a patient. The external transmitter inductive coil 335 is arranged coplanar to the internal receiver coil 331 so that it is in communication with the internal receiver coil 331 to provide wireless energy transfer to a device such as a VAD 301 implanted within the body. As shown in the schematic drawing of a CET system in FIG. 3B, the external components may also include a controller 319 and an external battery 313. The internal components include an implant (such as a VAD 301), a receiver inductive coil 331 coupled to the implant, an internal controller 309, and, optionally, an internal battery. The internal receiver coil 309 is placed within the body (for example, the pericardium sack) and receives power from the external transmission belt 345. The external transmission belt 345 of FIG. 3B can be disposed around an individual's torso like the external transmission belt 344 of FIG. 3A, arranged for transmitting power to an implant in the pericardium sack. The internal controller 309 controls power reception circuits, activates the implant electronics, and communicates with the external controller 319. The internal battery provides back-up power and enables operation of the implant independent of the wireless power transfer.

The transmission belt 345 includes a transmitter coil, and transmits power to the internal receiver coil 331 via a magnetic coupling. It is noted that a power source such as battery 313 must be associated with the external transmitter coil to provide that coil with the power that it will then wirelessly transmit for receipt by the implanted receiver coil 331. An external controller 319 regulates the operation of the transmitter coil. Like the transmitter coil, both the power source and the controller will be external to the patient. The external source can be an AC current source, and the transmitter coil can be electrically connected to the AC current source. It also is noted that the transmitter coil can be a transceiver—that is, capable of both transmitting and receiving. The external controller can run power transmission algorithms, communicate with the implant (e.g. through the frequency band of the Medical Implant Communication Service (MICS), which includes frequencies between 402 and 405 MHz), and push power to the belt from the battery 313. The external battery 313 is able to provide power to the transmission, and allow for generation of the electromagnetic field. Although FIGS. 3A and 3B depict the receiver ring implanted near the heart and the transmission belt configured to be placed around the abdomen, different configurations can be imagined, wherein the receiver is implanted in other parts of the patient's body such as the arm, leg, or head, and the transmission belt is correspondingly placed around that part of the body. Additional information about CET systems, components, and operation are described in US 2016/0233023 and US 2014/0031607, which are incorporated by reference in their entirety.

Wireless systems such as TET and CET are beneficial because they are less physically restrictive than wired systems and obviate the need for a transcutaneous wire. However, they present a safety concern due to their inaccessibility in case of emergency. If there is a failure in one of the implanted devices that prevents power from reaching the VAD, or if there are wireless connectivity problems, the patient may be at risk.

The hybrid powering systems of the present disclosure address many of these shortcomings while providing additional versatility to the patient. A hybrid powering system for an implanted medical device includes components for wireless power transmission, as well as a hardwired connection that can be used either as an emergency backup or for regular charging, or both. Emergency hardwire connections are described in US 2013/0053624, incorporated by reference in its entirety. With systems of the present disclosure however, a patient has several options for how to use the hardwired connection. The patient may choose, for example, to power up an implanted rechargeable battery using the hardwired connection any time it is convenient to do so, and then being free of any external apparatus for several hours; or alternatively, the patient may rely on the wireless assembly for everyday use and only use the hardwired connection in the even the wireless fails. Or a patient may enjoy the freedom of wireless powering when outside the home, but prefer the wired connection for home use or overnight charging. In any event, having two powering options gives the user freedom of choice. Rather than sacrificing convenience for safety, or vice versa, the hybrid powering system allows the patient to choose which type of power to use on an as-needed basis. The system also provides redundancy for the wireless power receiver, the implanted controller, or both.

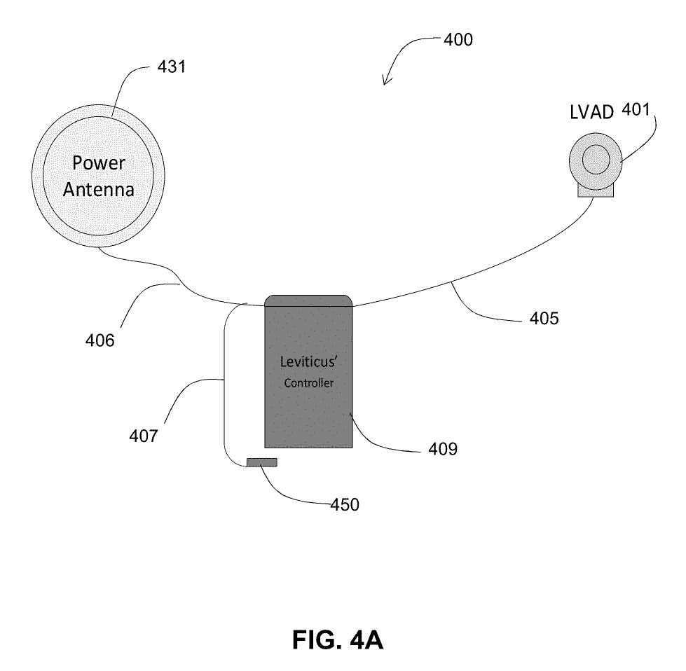

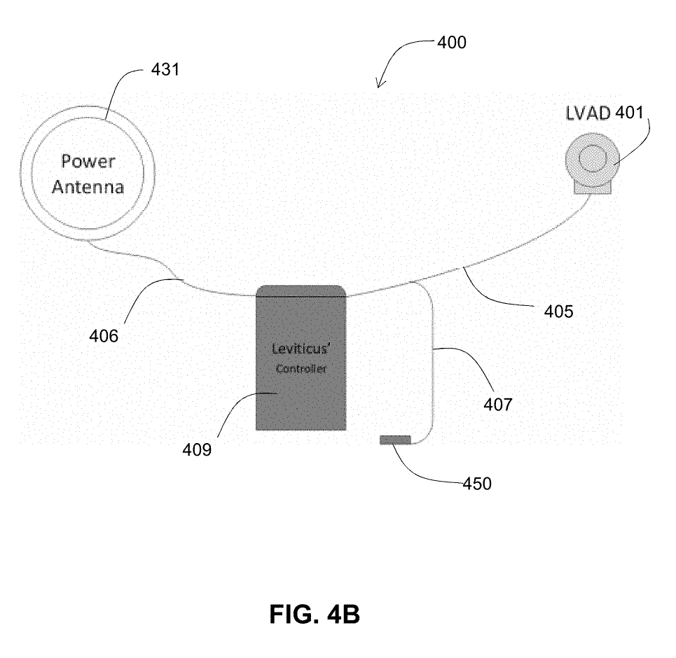

There are multiple ways to provide redundancy to the systems described herein. The two different embodiments shown in FIGS. 4A and 4B, which may be referred to a Type A and Type B, respectively, are both useful systems for providing redundancy to a wirelessly powered VAD system. The Type A system provides redundancy to the power that goes to the controller and charges the battery, which will be referred to as “input power.” The input power can be either AC or DC current or any other method of charging. Providing redundancy to the input power is the simpler and more straightforward configuration, but this Type A configuration lacks the ability to provide redundancy to the controller. The Type B system provides redundancy to the power that goes from the controller to the VAD, which will be referred to as “output power.” In certain embodiments the output power will be three-phase AC power that drives the VAD. Whereas the input power charges the battery, the output power is the power running from the controller to drive the VAD. Providing redundancy to the output power is more complex than providing redundancy to the input power, but it has the benefit of being able to back up to the entire system, not just the wireless receiver. A wired backup for the output power provides redundancy for the whole system because if there is a failure in the wireless receiver, the implanted controller, or the connections between them, the backup controller can step in and drive the VAD.

FIG. 4A shows a schematic depiction of components of a hybrid powering system 400 for an implanted medical device such as a VAD 401. As shown in the figure, the implanted components for receiving wireless power are supplemented by a hardwire connection. A left ventricular assist device, or LVAD, is connected by a wire 405 to an implantable controller 409 which includes a battery (not shown). Any type of VAD or any other implantable medical device is compatible with the invention, however. As shown, the implantable controller 409 is connected by another wire 406 to a power antenna 431, which in various configurations could be the receiver for a TET or a CET system as described above. In some embodiments the receiver 431 and the controller 409 are configured as a single unit. In a TET system the power antenna 431 would be implanted near the surface of the skin in a part of the patient's body. In a CET system, the power antenna 431 may be implanted elsewhere such as in in a pericardial sack in a coplanar arrangement with an external power transmission belt (not shown). These components of the system operate generally the same way as in the TET and CET systems described above. Generally speaking, electricity is wirelessly transmitted to the receiver 431, which in turn charges the battery in the controller 409.

In addition to the wireless power receiver 431, there is also a redundant external power source (not shown) that can be connected to the controller 409 by plugging in to the connector device 450 that leads to the controller 409 by wire 407. The connector device 450 generally takes the form of a socket that is configured to accept a plug (not shown) hardwired to a power source (also not shown) such as a battery or AC current source. The connector device 450 is configured to be implanted percutaneously in the patient with the socket oriented outward so that it is exposed external to the patient. The connector device 450 is thus electrically connected to the implanted VAD 401 while also being capable of receiving a plug from the external power source. The redundant power source is separate from the wireless power source. The two power sources can each have their own input connection to the controller, or they can be merged into a single wire, as is shown in FIG. 4A. In either configuration, however, the controller has two power inputs: a first one via the implanted receiver antenna 431 and a second one via a wire to an external power source. When the power source is plugged into the percutaneous connector device 450, a wired electrical connection is achieved between the external power source and the implanted medical device such as LVAD 401. The connector device 450 may be connected in parallel to the implanted controller 409 and the implanted power receiver 431. In this Type A configuration, the battery in the controller 409 can be charged by either of the two power sources.

Due to its percutaneous placement, the connector device 450 can be used for fast bailout of the implanted power receiver if it fails. The hardwired connection via connector device 450 provides a backup source for charging the battery, thereby reducing a risk associated with wireless powering. This allows backup input power without requiring surgery. This bailout could be performed by the patient or by a caregiver or any other individual nearby. But while this second source of input power in the Type A configuration provides redundancy for the wireless receiver, it does not provide redundancy for the whole system.

That is why it may be desirable in some situations to instead implement the more complex system shown in FIG. 4B, which provides redundancy for the implanted controller by including a second, external controller connectable to the VAD in parallel to the implanted controller. The second controller (not shown) is located external to the body and connects through a transcutaneous line and plugs into the VAD separately from the implanted controller. In some configurations these two sources of output power can be merged into a single wire, as is shown in FIG. 4B. The second controller thus provides a redundant source of output power. With redundancy for the output power, the system of FIG. 4B is able to provide backup to all of the implanted components. As will be described below, the system requires functionality for switching between the implanted controller and the external controller without risking a short in the VAD when two controllers are connected. Although this system is more complex than Type A, it is considered a more complete solution than input power redundancy, providing fuller protection to the user.

As with the Type A embodiment, in the Type B embodiment the wired connection occurs through the percutaneous connector. But with Type B, the connection provides controller redundancy as well as an external power source. This system is useful as a backup in the event that the internal controller fails, or simply to take over the operation of the VAD in emergency situations. The external controller may be separate from the external power source, or the two units may be coupled together as a single device.

In the Type B configuration shown in FIG. 4B, the VAD system has an output power line 405 leading from the internal controller to the VAD, and another output power line 407 leading from the transcutaneous connector device 450 located behind the ear. A redundant external controller (not shown) can be plugged into the connector device 450. The connector device 450 is depicted as being connected between the VAD 401 and the controller 409. This depiction is meant to show that the connector device 450 can provide redundancy to the controller 409 as well as just the wireless power receiver 431. However, it should be understood that the percutaneous connector device 450 can provide these functions regardless of the particular way that the components are arranged with respect to each other or within the body.

In any embodiment the redundant external controller may include a three-phase power supply connected in parallel to the internal controller 409. The external controller can drive the VAD 401 when the internal controller 409 has failed.

The disclosure recognizes that having two controllers simultaneously connected to the VAD introduces a potential complication into the system. That is, if both controllers were simultaneously providing output power to the VAD, it would result in a short or explosion. Therefore, the present invention provides a process for switching from internal power to external power without resulting in a short. The backup controller is configured to engage in a particular sequence of steps when it is connected, to ensure that the implanted controller is deactivated before the external controller begins providing the output power.

When the backup external controller is connected, it first senses whether the internal controller 409 is functional. If the internal controller 409 has stopped, the backup begins powering the VAD 401 and providing control from the external controller. However, if the internal controller is still providing output power when the backup is plugged in, the external controller will not drive the VAD 401 until the internal controller 409 has stopped. It will therefore communicate a signal to the internal controller to stop it from continuing to drive the VAD. The signal between the external controller and internal controller can be transmitted wirelessly via RF link such as through the MICS frequency band, or there can be a communication line that runs through the power lines that connect the controllers to the VAD, so that the signal can be sent via a high frequency communication signal modulation through the connected wire. The external controller then continues to validate whether the internal controller has stopped. It may do this by periodically pinging the internal controller to verify whether it is still providing output power. Once the external controller has confirmed that the internal controller has stopped, the external controller begins driving the VAD by sending output power through the hardwired connection.

The Type B configuration has been described as the more complex means of backing up the VAD system, because it requires the above-mentioned communication solution to prevent a short in the VAD. However, the Type A configuration may be preferred in some situations where a simpler or more robust or cost-effective system is desired.

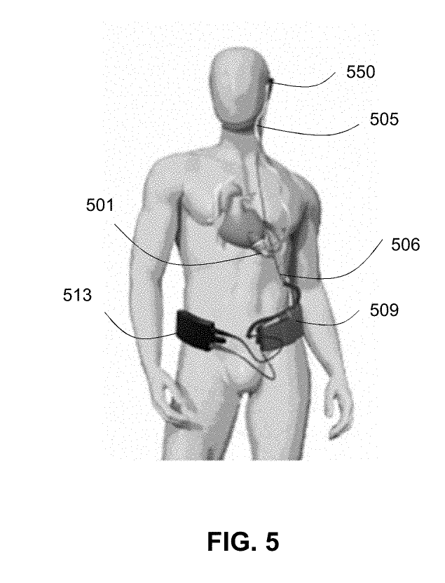

In some embodiments, the percutaneous connector device 450 is configured to be implanted behind the patient's ear. Using this region as the exit point for a percutaneous wire is considered to pose less risk of infection, compared with tunneling out of the abdomen as shown in FIG. 1. The connector device 450 can be surgically anchored into the osseous region behind the ear, and can be implanted in such a way as to limit the elements not covered by osseous proliferation. The soft tissue can be reduced to limit the incidence of infection. Devices compatible with the invention are described in US 2013/03030200, incorporated by reference herein. An example of a behind-ear configuration is shown in FIG. 5. The VAD 501 is connected by a transcutaneous wired connection that includes an implanted wire 505 connecting the VAD 501 to a percutaneously implanted connector device 550 implanted behind the patient's ear configured to receive a plug connected to an external wire 506 that connects with an external controller 509 and battery 513.

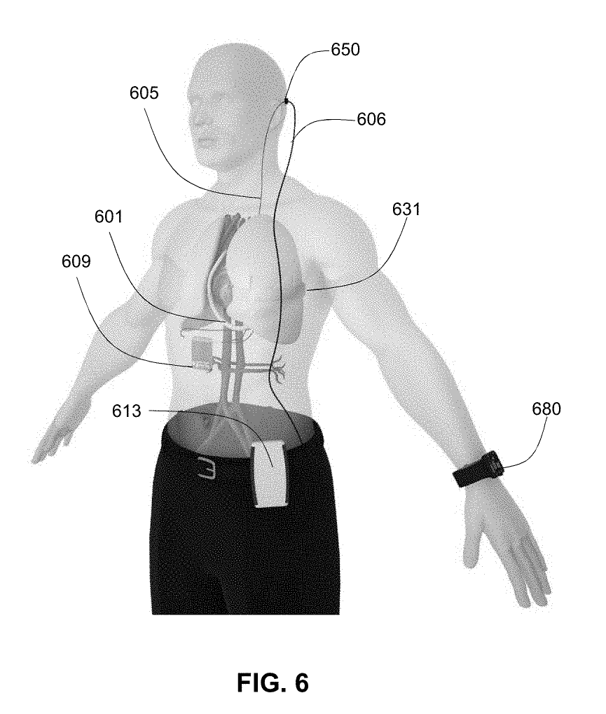

A preferred embodiment, however, is shown in FIG. 6, where like the prior embodiments the system includes a drive line connecting the VAD 601 to an internal controller 609 and a wireless power receiver 631, which in this case is shown as a CET power receiver coil disposed around the patient's lung. Another drive line or wire 605 connects the VAD 601 to the connector 650 located behind the ear. Here, the behind-the-ear connection serves as a backup to the wireless power transfer components. The connector device 650 is implanted percutaneously to provide a socket wherein an external power source 613 and/or controller can be plugged in. As shown, the external power source 613 is an external battery pack that the patient can wear on his belt and is available as a backup power source that can be plugged in to the connector device 650 by wire 606.

The hybrid power systems described herein can also include external communication devices for monitoring the function of the implanted VAD and power components. The external communication device can monitor various parameters of the implanted elements and alert the user to certain conditions, such as the need to recharge. Methods and systems for alerting a patient when an implanted battery is low are found in US 2015/0130283 and US 2018/0008760, which are incorporated herein by reference in their entirety. In some embodiments, the external communication device can take the form of a wristwatch or a tablet. An example of such a configuration is shown in FIG. 6. The patient has an implanted VAD 601, controller 609, and CET power receiver coil 631. An external battery pack 613 is shown plugged into the connector device 650 implanted percutaneously behind the ear. The patient has a wristwatch 680 configured to wirelessly communicate with the implanted components to alert the patient to certain conditions. In this configuration, the patient could unplug the external battery 613 when the wristwatch 680 indicated that the internal rechargeable battery (associated with the internal controller 609) was fully charged, and the patient could walk around with the only external apparatus being the wristwatch 680. The wristwatch 680 would then provide information such as remaining battery life and VAD function, to reassure the patient that his VAD implant was working properly. Once the implanted rechargeable battery begins running low, the wristwatch 680 would alert the patient that a power source is needed, whether it be a CET transmission belt (not shown) or plugging the external battery 613 into connector device 650 via wire 606. The wristwatch 680 may present other alerts as well, including amount of time remaining, and other parameters of the VAD. The wristwatch can replace the external controller function. It can provide wireless signals to the implanted controller using the MICS frequency band or another wireless protocol.

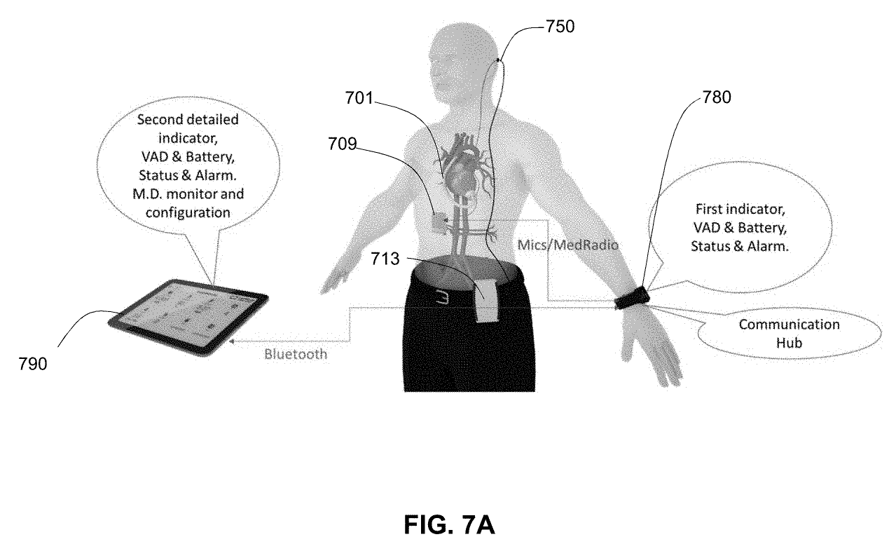

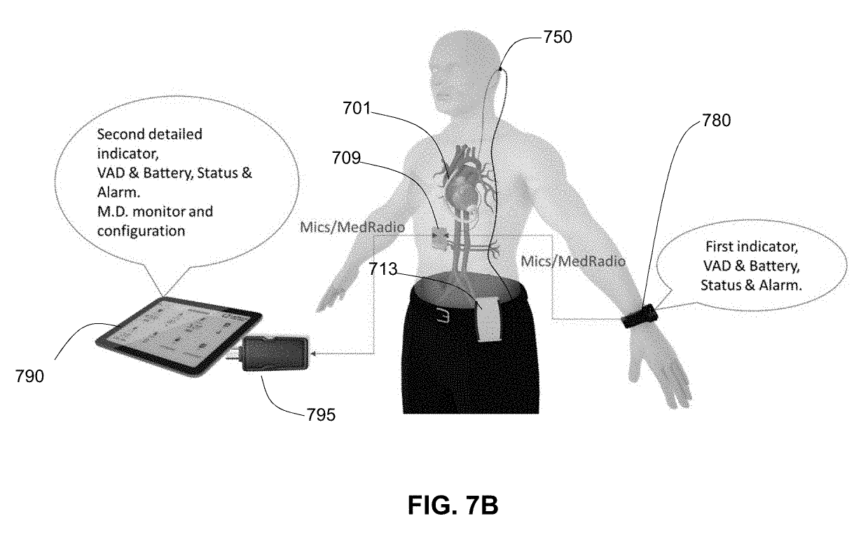

In some embodiments, the user control can be enhanced with the addition of a tablet, PC, or other device that can communicate with the wristwatch and/or directly with the implanted controller. FIGS. 7A and 7B show two configurations that employ a wirelessly connected tablet 790. FIG. 7A shows a VAD 701 and accompanying powering system as described above. An implanted controller 709 that includes a rechargeable battery controls the power and operation of the VAD 701. The controller 709 and battery can be separate, but are together in the embodiment shown. A drive line electrically connects the VAD 701 to a connector device 750 mounted behind the ear of the patient. An external battery 713 can be carried by the patient and is configured to be plugged into the connector device 750 to serve as an external power source to the VAD 701. A wristwatch 780 is configured to serve as a communication hub that can send and receive wireless signals to the implanted controller 709 via the MICS or MedRadio spectrum. The wristwatch 780 is configured to provide indicators of VAD performance, battery life, operational status, and alarms to alert the user to certain conditions. The wristwatch 780 may be similar to that described in published application US 2018/0126053, the contents of which are incorporated by reference. In addition to communicating with the implanted controller 709, the wristwatch 780 is configured to communicate with a tablet 790 or other similar device. The communication between the wristwatch 780 and the tablet 790 can use Bluetooth® or other similar wireless transmission modes. The tablet 790 can be a standard off-the-shelf tablet configurable to run an application for communicating with the wristwatch 780 using a common transmission mode. The tablet 790 can provide the same information and alerts as the wristwatch 780, as well as other usability features. For example, the patient, caregiver, or doctor can use the tablet 790 to monitor all of the relevant parameters of the implanted devices and change their configuration as needed.

As shown in FIG. 7B, in some embodiments the tablet 790 can communicate directly with the implanted controller 709. This may require a custom tablet configured to send and receive wireless transmissions in the MICS or MedRadio spectrum, since Bluetooth® is not the current industry standard for connectivity to implanted medical devices such as VADs, pacemakers, and implantable cardioverter-defibrillators. Alternatively, to convert an off-the-shelf tablet to enable communication with an implanted medical device, a dongle 795 can be provided that plugs into the tablet 790 (or personal computer or other monitoring device) with a standard wired protocol such as USB. The dongle 795 can wirelessly connect with the implanted controller 709 and communicate with the tablet 790 using the wired protocol, while also receiving power from its host device (such as tablet 790) through the same wired protocol, or it can be self-powered. Other similar permutations of the system could be readily imagined by the person of ordinary skill in the art. In any case, the invention contemplates a wireless device that bridges the two wireless protocols (MICS and Bluetooth® or other standard protocol used by off-the-shelf tablets or PCs) to allow communication from the implanted medical devices to a device such as a wristwatch or a dongle, and ultimately to a display device such as a tablet or PC.

What is claimed is:

1. A system for treating a heart condition in a patient, the system comprising:
a ventricular assist device (VAD) configured to be implanted within a body of a patient;
a controller configured to be implanted within the body and coupled to the VAD, the controller configured to drive the VAD;
a power receiver configured to be implanted within the body and coupled to the implanted controller, the implanted power receiver configured to wirelessly receive electromagnetic power from a power transmitter disposed external to the body, and further configured to provide power to the controller; and
a connector device configured to be implanted percutaneously in the body and hardwired to the controller, the connector device further configured to be hardwired to a power source external to the patient to provide redundancy for the implanted power receiver.

2. The system of claim 1, wherein the implanted controller comprises a rechargeable battery.

3. The system of claim 2, wherein the rechargeable battery is configured to be charged by one or both of the implanted power receiver and the external power source.

4. The system of claim 1, further comprising an external monitoring device configured to wirelessly communicate with the implanted controller.

5. The system of claim 4, wherein the external monitoring device is configured to receive data from the implanted controller, the data comprising an operational status of the VAD.

6. The system of claim 5, wherein the external monitoring device further comprises a display configured to display the data.

7. The system of claim 1, wherein the connector device is configured to be disposed behind the patient's ear.

8. The system of claim 1, wherein the connector device comprises a socket configured to accept a plug that is hardwired to the external power source.

9. The system of claim 1, wherein the implanted power receiver is configured to be disposed under an area of the patient's skin and to receive transcutaneous power when the power transmitter is located adjacent to the area of the patient's skin.

10. The system of claim 1, wherein the implanted power receiver comprises a receiver inductive coil and wherein the power transmitter comprises a transmitter inductive coil disposed external to the patient and surrounding the receiver inductive coil.

11. A system for treating a heart condition in a patient, the system comprising:
a ventricular assist device (VAD) configured to be implanted within a body of a patient;
a controller configured to be implanted within the body and coupled to the VAD, the controller configured to drive the VAD;
a power receiver configured to be implanted within the body and coupled to the implanted controller, the implanted power receiver configured to wirelessly receive electromagnetic power from a power transmitter disposed external to the body, and further configured to provide power to the controller; and
a connector device configured to be implanted percutaneously in the body and hardwired to the VAD, the connector device further configured to be hardwired to a controller external to the patient, the external controller configured to determine whether the implanted controller is driving the VAD and to provide redundancy for the implanted controller only when the implanted controller is not driving the VAD.

12. The system of claim 11, wherein the external controller is associated with an external power source.

13. The system of claim 11, wherein the external controller is configured to communicate a signal to the implanted controller to cause the implanted controller to stop driving the VAD.

14. The system of claim 13, wherein the signal is transmitted wirelessly or via a communication line that runs through the power lines that connect the controllers to the VAD.

15. The system of claim 13, wherein the signal is sent in response to the external controller being plugged in to the connector device.

16. The system of claim 11, further comprising an external monitoring device configured to wirelessly communicate with the implanted controller.

17. The system of claim 16, wherein the external monitoring device is configured to receive data from the implanted controller, the data comprising an operational status of the VAD.

18. The system of claim 17, wherein the external monitoring device further comprises a display configured to display the data.

19. The system of claim 11, wherein the connector device is configured to be disposed behind the patient's ear.

20. The system of claim 11, wherein the connector device comprises a socket configured to accept a plug that is hardwired to the external controller.
